(19) United States
(12) Patent Application Publication
(10) Pub. No.: US 2025/0339436 A1
(43) Pub. Date: Nov. 6, 2025

(54) CRYSTALLINE FORMS OF MRTX1133

(71) Applicant: MIRATI THERAPEUTICS, INC., PRINCETON, NJ (US)

(72) Inventors: PATRICIA ANDRES, SAN DIEGO, CA (US); TAWFIK GHARBAOUI, SAN DIEGO, CA (US)

(73) Assignee: MIRATI THERAPEUTICS, INC., PRINCETON, NJ (US)

(21) Appl. No.: 19/196,912

(22) Filed: May 2, 2025

Related U.S. Application Data
(60) Provisional application No. 63/642,498, filed on May 3, 2024.

(51) Int. Cl. A61K 31/519; C07D 519/00

(52) U.S. Cl. CPC A61K 31/519; C07D 519/00

(57) ABSTRACT

The present invention relates to crystalline forms of MRTX1133 (4-(4-((1R,5S)-3,8-diazabicyclo[3.2.1]octan-3-yl)-8-fluoro-2-(((2R,7aS)-2-fluorohexahydro-1H-pyrrolizin-7a-y1) methoxy)pyrido[4,3-d]pyrimidin-7-yl)-5-ethynyl-6-fluoronaphthalen-2-ol), pharmaceutical compositions comprising the crystalline forms, processes for preparing the crystalline forms and methods of use thereof.

### CROSS REFERENCE TO RELATED APPLICATIONS

This application claims the benefit of U.S. Provisional Application No. 63/642,498, filed May 3, 2024, the entire content of which is hereby incorporated herein by reference.

### FIELD OF THE INVENTION

The present invention relates to crystalline forms of MRTX1133 (4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol); pharmaceutical compositions comprising the crystalline forms, processes for preparing the crystalline forms and methods of use thereof.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13 (12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma. KRAS G12D mutation is present in 25.0% of all pancreatic ductal adenocarcinoma patients, 13.3% of all colorectal carcinoma patients, 10.1% of all rectal carcinoma patients, 4.1% of all non-small cell lung carcinoma patients and 1.7% of all small cell lung carcinoma patients (e.g., see The AACR Project GENIE Consortium, (2017) Cancer Discovery; 7 (8): 818-831. Dataset Version 4).

The well-known role of KRas in malignancy and the discovery of these frequent mutations in KRas in various tumor types made KRas a highly attractable target of the pharmaceutical industry for cancer therapy.

Compounds that inhibit KRas activity are still highly desirable and under investigation, including those that disrupt effectors such as guanine nucleotide exchange factors (e.g., see Sun et al., (2012) Agnew Chem Int Ed Engl. 51 (25): 6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly, there remains a continued interest and effort to develop inhibitors of KRas, particularly inhibitors of activating KRas mutants, especially KRas G12D.

A noncovalent inhibitor of KRas G12D is MRTX1133 (4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol). An amorphous form of this compound was described in International Patent Application PCT/US2020/048194 filed Aug. 27, 2020 and published as WIPO publication WO2021/041671 on Mar. 4, 2021 at Example 252. The compound is also described in Qinheng Zheng et al, Identification of MRTX1133, a Noncovalent, Potent, and Selective KRAS^{G12D} Inhibitor, J. Med. Chem, 2022, 65, 4, 3123-3133.

For all the foregoing reasons, there is a need to produce a solid, crystalline form of MRTX1133, that would ideally provide enhanced chemical stability, dissolution rate, solubility, bioavailability, manufacturing improvements and/or storage shelf life of the pharmaceutical composition. The present invention advantageously addresses one or more of those needs.

### SUMMARY OF THE INVENTION

In one aspect of the invention, provided herein are crystalline forms of the KRas G12D inhibitor MRTX1133 (4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol).

In one embodiment, the crystalline form is crystalline Form VIII. In one embodiment, crystalline Form VIII has an X-ray powder diffraction pattern (“XRPD”) comprising at least one characteristic peak at °2θvalues selected from 11.1±0.3, 13.4±0.3, 14.3±0.3, 19.1±0.3, 22.9±0.3, and 19.8±0.3. In some embodiments only a single characteristic peak is present. In some embodiments two characteristic peaks are present. In some embodiments three characteristic peaks are present. In some embodiments four characteristic peaks are present. In some embodiments five characteristic peaks are present. In some embodiments six characteristic peaks are present.

In one embodiment, crystalline Form VIII has an X-ray powder diffraction pattern comprising peaks at °2θvalues of 11.1±0.3, 14.3±0.3, and 19.1±0.3.

In another embodiment, crystalline Form VIII has an X-ray powder diffraction pattern comprising two or more peaks at °2θ at 11.1±0.3, 13.4±0.3, 14.3±0.3, 19.1±0.3, 22.9±0.3, and 19.8±0.3.

In another embodiment, crystalline Form VIII has an X-ray powder diffraction pattern comprising three or more peaks at °2θ at 11.1±0.3, 13.4±0.3, 14.3±0.3, 19.1±0.3, 22.9±0.3, and 19.8±0.3.

In other embodiments, crystalline Form VIII has an XRPD pattern substantially as shown in FIG. 1.

In one embodiment, crystalline Form VIII is characterized by a differential scanning calorimetry (“DSC”) thermogram substantially as shown in FIG. 2.

In another embodiment, crystalline Form VIII has both: 1) a DSC characteristic described above; and 2) an X-ray powder diffraction pattern comprising at least one peak at 20 selected from 11.1±0.3, 13.4±0.3, 14.3±0.3, 19.1±0.3, 22.9±0.3, and 19.8±0.3.

In another embodiment, crystalline Form VIII has both: 1) a DSC characteristic described above; and 2) an X-ray powder diffraction pattern comprising peaks at °2θvalues of 11.1±0.3, 14.3±0.3, and 19.1±0.3

In one embodiment, crystalline Form VIII is characterized by having about 0.7% weight loss until the onset of degradation at about 180° C. as estimated by thermogravimetric analysis (“TGA”). In another embodiment, crystalline Form VIII has a TGA profile substantially as shown in FIG. 3.

In another embodiment, crystalline Form VIII has both: 1) one or more TGA characteristics described above; and 2) an X-ray powder diffraction pattern comprising at least one peak at 020 selected from 11.1±0.3, 13.4±0.3, 14.3±0.3, 19.1±0.3, 22.9±0.3, and 19.8±0.3. In another embodiment, crystalline Form VIII has both: 1) one or more TGA characteristics described above; and 2) an X-ray powder diffraction pattern comprising peaks at °2θvalues of 11.1±0.3, 14.3±0.3, and 19.1±0.3.

In one embodiment, crystalline Form VIII is characterized by having an observed water uptake of about 1.3% at 25° C./95% Relative Humidity (RH), as measured by dynamic vapor sorption (“DVS”).

In another embodiment, crystalline Form VIII has a DVS isotherm substantially as shown in FIG. 4.

In another embodiment, crystalline Form VIII has both: 1) one or more DVS characteristics described above; and 2) an X-ray powder diffraction pattern comprising at least one peak at 020 selected from 11.1±0.3, 13.4±0.3, 14.3±0.3, 19.1±0.3, 22.9±0.3, and 19.8±0.3.

In another embodiment, crystalline Form VIII has both: 1) one or more DVS characteristics described above; and 2) an X-ray powder diffraction pattern comprising peaks at °2θvalues of 11.1±0.3, 14.3±0.3, and 19.1±0.3.

In one embodiment, crystalline Form VIII has a ¹H-NMR profile substantially as shown in FIG. 5.

In one embodiment, crystalline Form VIII is substantially free of residual organic solvents.

In one embodiment, the crystalline form is designated crystalline Form IX.

In one embodiment, crystalline Form IX has an XRPD pattern comprising at least one characteristic peak at °2θvalues selected from 5.5±0.3, 10.9±0.3, 14.0±0.3, 17.4±0.3, 21.7±0.3, and 24.0±0.3. In some embodiments only a single characteristic peak is present. In some embodiments two characteristic peaks are present. In some embodiments three characteristic peaks are present. In some embodiments four characteristic peaks are present. In some embodiments five characteristic peaks are present. In some embodiments six characteristic peaks are present.

In one embodiment, crystalline Form IX has an XRPD pattern comprising peaks at °2θvalues of 5.5±0.3, 10.9±0.3, and 21.7±0.3.

In another embodiment, crystalline Form IX has an XRPD pattern comprising two or more peaks at °2θ at 5.5±0.3, 10.9±0.3, 14.0±0.3, 17.4±0.3, 21.7±0.3, and 24.0±0.3.

In another embodiment, crystalline Form IX has an XRPD pattern comprising three or more peaks at °2θ at 5.5±0.3, 10.9±0.3, 14.0±0.3, 17.4±0.3, 21.7±0.3, and 24.0±0.3.

In other embodiments, crystalline Form IX has an XRPD pattern substantially as shown in FIG. 6A or FIG. 6B.

In one embodiment, crystalline Form IX is characterized by having a DSC thermogram substantially as shown in FIG. 7.

In another embodiment, crystalline Form IX has both: 1) a DSC characteristic described above; and 2) an X-ray powder diffraction pattern comprising at least one peak at °2θ selected from 5.5±0.3, 10.9±0.3, 14.0±0.3, 17.4±0.3, 21.7±0.3, and 24.0±0.3.

In another embodiment, crystalline Form IX has both: 1) a DSC characteristic described above; and 2) an XRPD pattern comprising peaks at °2θvalues of 5.5±0.3, 10.9±0.3, and 21.7±0.3.

In one embodiment, crystalline Form IX is characterized by having about 0.5% weight loss until the onset of degradation at about 190° C. as estimated by TGA. In another embodiment, crystalline Form IX has a TGA profile substantially as shown in FIG. 8.

In another embodiment, crystalline Form IX has both: 1) one or more TGA characteristics described above; and 2) an XRPD pattern comprising at least one peak at °2θ selected from 5.5±0.3, 10.9±0.3, 14.0±0.3, 17.4±0.3, 21.7±0.3, and 24.0±0.3. In another embodiment, crystalline Form IX has both: 1) one or more TGA characteristics described above; and 2) an XRPD pattern comprising peaks at °2θvalues of 5.5±0.3, 10.9±0.3, and 21.7±0.3.

In one embodiment, crystalline Form IX has a DVS isotherm substantially as shown in FIG. 9.

In another embodiment, crystalline Form IX has both: 1) a DVS characteristic described above; and 2) an XRPD pattern comprising at least one peak at 20 selected from 5.5±0.3, 10.9±0.3, 14.0±0.3, 17.4±0.3, 21.7±0.3, and 24.0±0.3.

In another embodiment, crystalline Form IX has both: 1) a DVS characteristic described above; and 2) an XRPD pattern comprising peaks at °2θvalues of 5.5±0.3, 10.9±0.3, and 21.7±0.3.

In one embodiment, crystalline Form IX has a ¹H-NMR profile substantially as shown in FIG. 10.

In one embodiment, crystalline Form IX is substantially free of residual organic solvents.

In one embodiment, the crystalline forms of the present invention are at least 40%, 50%, 60%, 70%, 80%, 90% or 95% crystalline.

In another aspect of the invention, pharmaceutical compositions are provided for use in the methods comprising a therapeutically effective amount of at least one of the following: crystalline Form VIII and crystalline Form IX, and/or a pharmaceutically acceptable excipient. The invention also encompasses pharmaceutical compositions comprising any of other crystalline forms described in the application.

The invention also encompassed any mixtures of any of the described crystalline forms with the amorphous form of MRTX1133.

In some embodiments, the pharmaceutical compositions of the present invention comprise at least 95%, or at least 80%, or at least 70%, or at least 60%, or at least 50% of crystalline Form VIII of MRTX1133.

In some embodiments, the pharmaceutical compositions of the present invention comprise at least 95%, or at least 80%, or at least 70%, or at least 60%, or at least 50% of crystalline Form IX of MRTX1133.

In one aspect of the invention, provided herein are methods for inhibiting KRas G12D activity in a cell, comprising contacting the cell in which inhibition of KRas G12D activity is desired with a therapeutically effective amount of a crystalline form of the present invention, alone or in combination with one or more pharmaceutically acceptable excipients and/or diluents. In one embodiment, the crystalline form is crystalline Form VIII. In another embodiment, the crystalline form is crystalline Form IX. In other embodiments, the crystalline form is any of the other forms described in this application.

In one embodiment, the crystalline form is a mixture of crystalline Form VIII and crystalline Form IX. In another embodiment, the crystalline form is a mixture of any of the described crystalline forms with the amorphous form.

In one aspect of the invention, provided herein are methods of treating cancer in a subject in need thereof, comprising administering to the subject a therapeutically effective amount of a crystalline form of MRTX1133. In one embodiment, the cancer is a KRas G12D-associated cancer. In one embodiment, the KRas G12D-associated cancer is lung cancer.

In one embodiment, the crystalline form is crystalline Form VIII. In another embodiment, the crystalline form is crystalline Form IX. In other embodiments, the crystalline form is any of the other forms described in this application.

In one embodiment, the crystalline form is a mixture of crystalline Form VIII and crystalline Form IX. In another embodiment, the crystalline form is a mixture of any of the described crystalline forms with the amorphous form.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a crystalline form of MRTX1133, alone or in combination with one or more pharmaceutically acceptable excipients and/or diluent.

In one embodiment, the subject is an adult patient. In one embodiment, the subject is a pediatric patient.

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

In another aspect of the invention, provided herein are process for the preparation of crystalline forms of MRTX1133. In one embodiment, the process describes the preparation of crystalline Form VIII. In one embodiment, the process describes the preparation of crystalline Form IX. In other embodiments, the process describes the preparation of other crystalline forms of MRTX1133.

### BRIEF DESCRIPTION OF THE FIGURES

FIG. 1A illustrates X-ray powder diffraction (XRPD) pattern of crystalline Form VIII of MRTX1133, prepared according to the methods described in the application.

FIG. 1B illustrates another XRPD pattern of crystalline Form VIII of MRTX1133, prepared according to the methods described in the application.

FIG. 2 illustrates a differential scanning calorimetry (DSC) profile of crystalline Form VIII prepared according to the methods described in the application.

FIG. 3 illustrates a thermogravimetric analysis (TGA) profile of crystalline Form VIII prepared according to the methods described in the application.

FIG. 4 illustrates a dynamic vapor sorption (DVS) isotherm profile of crystalline Form VIII prepared according to the methods described in the application.

FIG. 5 illustrates a solution ¹H-NMR profile of crystalline Form VIII prepared according to the methods described in the application.

FIG. 6A illustrates an XRPD pattern of crystalline Form IX prepared according to the methods described in the application.

FIG. 6B illustrates another XRPD pattern of crystalline Form IX of MRTX1133, prepared according to the methods described in the application.

FIG. 7 illustrates a DSC profile of crystalline Form IX prepared according to the methods described in the application.

FIG. 8 illustrates a TGA profile of crystalline Form IX prepared according to the methods described in the application.

FIG. 9 illustrates a DVS isotherm profile of crystalline Form IX prepared according to the methods described in the application.

FIG. 10 illustrates a solution ¹H-NMR profile of crystalline Form IX prepared according to the methods described in the application.

FIG. 11 illustrates an XRPD pattern of crystalline Form II prepared according to the methods described in the application.

FIG. 12 illustrates an XRPD pattern of crystalline Form VI prepared according to the methods described in the application.

FIG. 13 illustrates an XRPD pattern of crystalline Form I prepared according to the methods described in the application.

FIG. 14 illustrates an XRPD pattern of crystalline Form VII prepared according to the methods described in the application.

### DETAILED DESCRIPTION OF THE INVENTION

The present invention relates to crystalline forms of the KRas G12D inhibitor MRTX1133 (4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol). In particular, the present invention relates to crystalline Form VIII and crystalline Form IX, pharmaceutical compositions comprising the crystalline forms, processes for preparing the crystalline forms and methods of use thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp.Glyl2Asp.

As used herein, a “KRas G12D inhibitor” refers to compounds of the present invention that are represented by Formula (I), as described herein. These compounds are capable of negatively modulating or inhibiting all or a portion of the enzymatic activity of KRas G12D.

A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, the term “solvate” refers to a crystalline form of MRTX1133 which contains solvent.

As used herein, the term “hydrate” refers to a solvate wherein the solvent comprises water.

As used herein, the term “residual organic solvents” refers to organic volatile chemicals used or produced during the crystallization/manufacturing processes that are not completely removed during the manufacturing technique.

As used herein, the term “substantially free of residual organic solvents” means that the manufactured pharmaceutical preparation, e.g., a pharmaceutical preparation comprising a crystalline form of MRTX1133, contains less than 1.0% by weight of residual organic solvents, contains less than 0.5% by weight of residual organic solvents, contains less than 0.4% by weight of residual organic solvents, contains less than 0.3% by weight of residual organic solvents, contains less than 0.2% by weight of residual organic solvents, or contains less than 0.1% by weight of residual organic solvents.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12D mutation (e.g., as determined using a regulatory agency-approved, e.g., FDA-approved, assay or kit). In some embodiments, the subject has a tumor that is positive for a KRas G12D mutation (e.g., as determined using a regulatory agency-approved assay or kit). The subject can be a subject with a tumor(s) that is positive for a KRas G12D mutation (e.g., identified as positive using a regulatory agency-approved, e.g., FDA-approved, assay or kit). The subject can be a subject whose tumors have a KRas G12D mutation (e.g., where the tumor is identified as such using a regulatory agency-approved, e.g., FDA-approved, kit or assay). In some embodiments, the subject is suspected of having a KRas G12D gene-associated cancer. In some embodiments, the subject has a clinical record indicating that the subject has a tumor that has a KRas G12D mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has a KRas G12D mutation using a sample (e.g., a biological sample or a biopsy sample such as a paraffin-embedded biopsy sample) from a patient (e.g., a patient suspected of having a KRas G12D-associated cancer, a patient having one or more symptoms of a KRas G12D-associated cancer, and/or a patient that has an increased risk of developing a KRas G12D-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, a “therapeutically effective amount” of a crystalline form of MRTX1133 is an amount that is sufficient to ameliorate, or in some manner reduce a symptom or stop or reverse progression of a condition, or negatively modulate or inhibit the activity of KRas G12D. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of a crystalline form of MRTX1133, or the length of treatment time described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

As used herein, the term “about” when used in reference to XRPD peak positions refers to the inherent variability of peaks depending on the calibration of the instrument, processes used to prepare the crystalline forms of the present invention, age of the crystalline forms and the type of instrument used in the analysis. The variability of the instrumentation used for XRPD analysis is about ±0.2° 2θ. Variability of peaks of MRTX1133 was observed to be ±0.3 °2θ however, as is known to happen for variable solvates and in some instances non-solvated forms derived from solvates and/or hydrates.

### GENERAL METHODS AND INSTRUMENTATION

The general methods outlined below were used in the exemplified Examples, unless otherwise noted.

Crystalline forms may be analyzed using any suitable analytical method or assay procedure including, but not limited to, X-Ray Powder Diffraction, NMR, differential scanning calorimetry, thermo-gravimetric analysis, and gravimetric vapor sorption to assure formation of the preferred crystalline form of MRTX1133. The crystalline form is typically produced in an amount of greater that 50% by weight isolated yield, greater that 60% by weight isolated yield, greater that 70% by weight isolated yield, greater that 80% by weight isolated yield, greater that 90% by weight isolated yield or greater that 95% by weight isolated yield. In one embodiment, the crystalline forms of the present invention are at least 40%, 50%, 60%, 70%, 80%, 90% or 95% crystalline.

### Instruments and Methods

### X-ray Powder Diffraction (XRPD)

### Instrument: Bruker D8 Advance

Parameters: X-Ray tube Cu (Kα radiation); tube voltage 40 kV; tube current 40 mA
Scanning range: 3 to 40 2θ (degree)
Step size: 0.02 degree
Scanning speed: 0.12 second per step
or

### Instrument: Rigaku MiniFlex 300/600

Parameters: X-Ray tube Cu (Kα radiation); tube voltage 40 kV; tube current 15 mA
Scanning range: 3 to 40 2θ (degree)
Step width: 0.02 degree
Scanning speed: 10.00°/min

### Thermogravimetric Analysis (TGA)

### Instrument: Discovery 5500

Parameters: Nitrogen Flow balance 10 mL/min; sample 25 mL/min; start temperature: ambient condition (below 35° C.); final temperature: 300° C.; heating rate: 10° C./min; sample mass ˜2-10 mg.
Differential Scanning calorimetry (DSC)

### Instrument: Discovery 2500

Parameters: temperature range: 30 to 250° C.; heating rate: 10° C./min; nitrogen flow: 50 mL/min; sample mass ˜1-2 mg.

### Dynamic Vapor Sorption (DVS)

### Instrument: Intrinsic, Advantage or Adventure;

Total gas flow: 200 sccm; oven temperature: 25° C., solvent: water; method:
Cycle: 40-0-95-0-40% RH
Stage Step: 10%
Equilibrium: 0.002 dm/dt (%/min) Minimum dm/dt stability duration: 60 min
Maximum dm/dt stage time: 360 min

### Nuclear Magnetic Resonance

Solution proton NMR spectra were acquired using a Bruker Advance-AV 400M spectrometer, frequency 400 MHz, according to the manufacturer's instructions. Probe: 5 mm PABBO BB-1H/D; number of scan: 8; temperature: 297.6K; relaxation delay: 1 second.

### KRAS G12D Inhibitor

In one aspect of the invention, provided herein are crystalline forms of MRTX1133, also known as 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol.

Methods for manufacturing MRTX1133 are known. For example, MRTX1133 is described in Example 252 of PCT Application WO 2021/041671.

### Crystalline Forms of Mrtx1133

In one embodiment, the crystalline form is crystalline Form VIII. In one embodiment, crystalline Form VIII has an X-ray powder diffraction pattern (“XRPD”) comprising at least one characteristic peak at °2θvalues selected from 11.1±0.3, 13.4±0.3, 14.3±0.3, 19.1±0.3, 22.9±0.3, and 19.8±0.3. In some embodiments only a single characteristic peak is present. In some embodiments two characteristic peaks are present. In some embodiments three characteristic peaks are present. In some embodiments four characteristic peaks are present. In some embodiments five characteristic peaks are present. In some embodiments six characteristic peaks are present.

In one embodiment, crystalline Form VIII has an X-ray powder diffraction pattern comprising peaks at °2θvalues of 11.1±0.3, 14.3±0.3, and 19.1±0.3. In another embodiment, crystalline Form VIII has an X-ray powder diffraction pattern comprising two or more peaks at °2θ at 11.1±0.3, 13.4±0.3, 14.3±0.3, 19.1±0.3, 22.9±0.3, and 19.8±0.3.

In another embodiment, crystalline Form VIII has an X-ray powder diffraction pattern comprising three or more peaks at °2θ at 11.1±0.3, 13.4±0.3, 14.3±0.3, 19.1±0.3, 22.9±0.3, and 19.8±0.3.

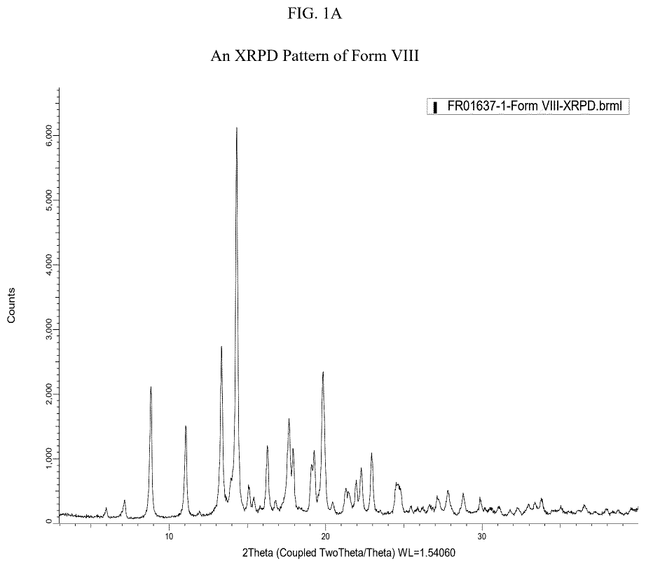

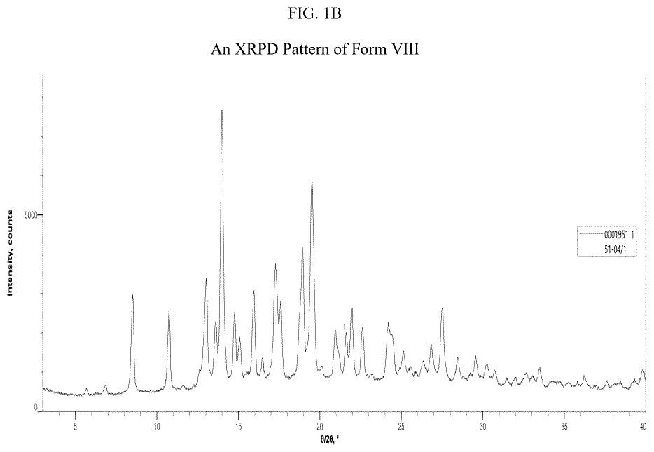

In other embodiments, crystalline Form VIII has an XRPD pattern substantially as shown in FIG. 1A or FIG. 1B.

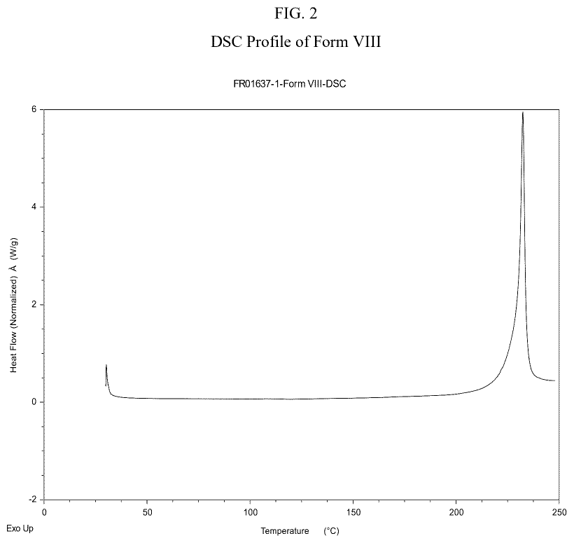

In one embodiment, crystalline Form VIII is characterized by a differential scanning calorimetry (“DSC”) thermogram substantially as shown in FIG. 2.

In another embodiment, crystalline Form VIII has both: 1) a DSC characteristic described above; and 2) an X-ray powder diffraction pattern comprising at least one peak at °2θ selected from 11.1±0.3, 13.4±0.3, 14.3±0.3, 19.1±0.3, 22.9±0.3, and 19.8±0.3.

In another embodiment, crystalline Form VIII has both: 1) a DSC characteristic described above; and 2) an X-ray powder diffraction pattern comprising peaks at 20 values of 11.1±0.3, 14.3±0.3, and 19.1±0.3.

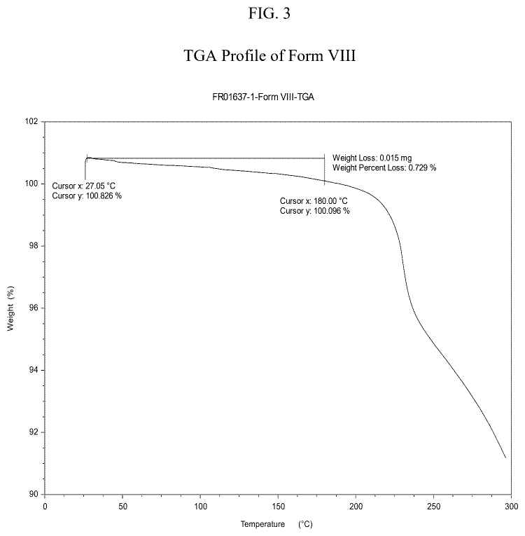

In one embodiment, crystalline Form VIII is characterized by having about 0.7% weight loss until the onset of degradation at about 180° C. as estimated by thermogravimetric analysis (“TGA”). In another embodiment, crystalline Form VIII has a TGA profile substantially as shown in FIG. 3.

In another embodiment, crystalline Form VIII has both: 1) one or more TGA characteristics described above; and 2) an X-ray powder diffraction pattern comprising at least one peak at 020 selected from 11.1±0.3, 13.4±0.3, 14.3±0.3, 19.1±0.3, 22.9±0.3, and 19.8±0.3. In another embodiment, crystalline Form VIII has both: 1) one or more TGA characteristics described above; and 2) an X-ray powder diffraction pattern comprising peaks at °2θvalues of 11.1±0.3, 14.3±0.3, and 19.1±0.3.

In one embodiment, crystalline Form VIII is characterized by having an observed water uptake of about 1.3% at 25° C./95% Relative Humidity (RH), as measured by dynamic vapor sorption (“DVS”).

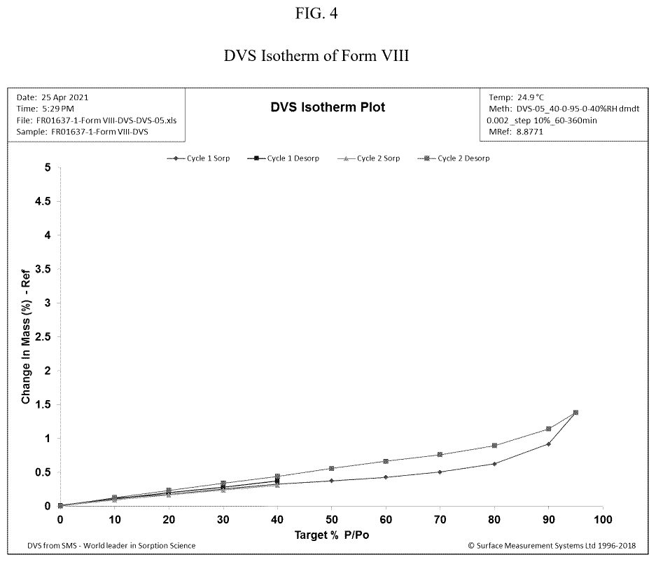

In another embodiment, crystalline Form VIII has a DVS isotherm substantially as shown in FIG. 4.

In another embodiment, crystalline Form VIII has both: 1) one or more DVS characteristics described above; and 2) an X-ray powder diffraction pattern comprising at least one peak at 020 selected from 11.1±0.3, 13.4±0.3, 14.3±0.3, 19.1±0.3, 22.9±0.3, and 19.8±0.3.

In another embodiment, crystalline Form VIII has both: 1) one or more DVS characteristics described above; and 2) an X-ray powder diffraction pattern comprising peaks at °2θvalues of 11.1±0.3, 14.3±0.3, and 19.1±0.3.

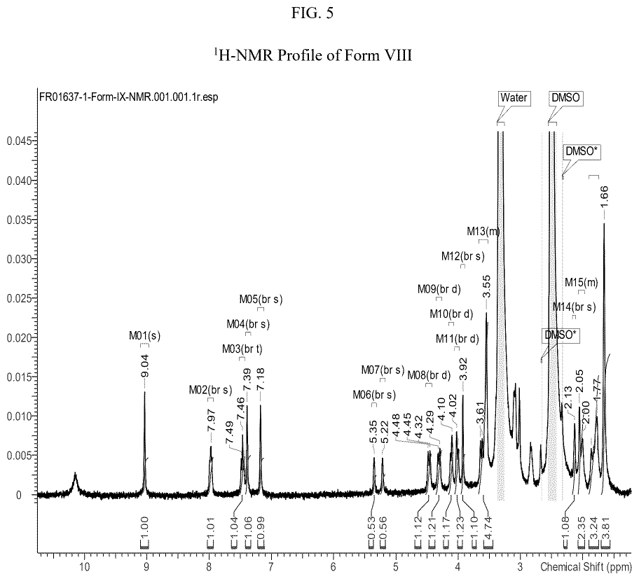

In one embodiment, crystalline Form VIII has a ¹H-NMR profile substantially as shown in FIG. 5.

In one embodiment, crystalline Form VIII is substantially free of residual organic solvents.

In one embodiment, the crystalline form is designated crystalline Form IX.

In one embodiment, crystalline Form IX has an XRPD pattern comprising at least one characteristic peak at °2θvalues selected from 5.5±0.3, 10.9±0.3, 14.0±0.3, 17.4±0.3, 21.7±0.3, and 24.0±0.3. In some embodiments only a single characteristic peak is present. In some embodiments two characteristic peaks are present. In some embodiments three characteristic peaks are present. In some embodiments four characteristic peaks are present. In some embodiments five characteristic peaks are present. In some embodiments six characteristic peaks are present.

In one embodiment, crystalline Form IX has an XRPD pattern comprising peaks at °2θvalues of 5.5±0.3, 10.9±0.3, and 21.7±0.3.

In another embodiment, crystalline Form IX has an XRPD pattern comprising two or more peaks at °2θ at 5.5±0.3, 10.9±0.3, 14.0±0.3, 17.4±0.3, 21.7±0.3, and 24.0±0.3.

In another embodiment, crystalline Form IX has an XRPD pattern comprising three or more peaks at °2θ at 5.5±0.3, 10.9±0.3, 14.0±0.3, 17.4±0.3, 21.7±0.3, and 24.0±0.3.

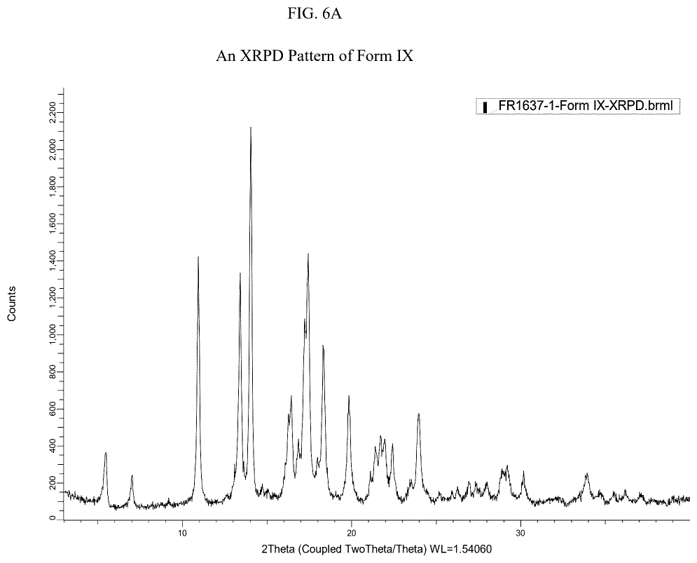

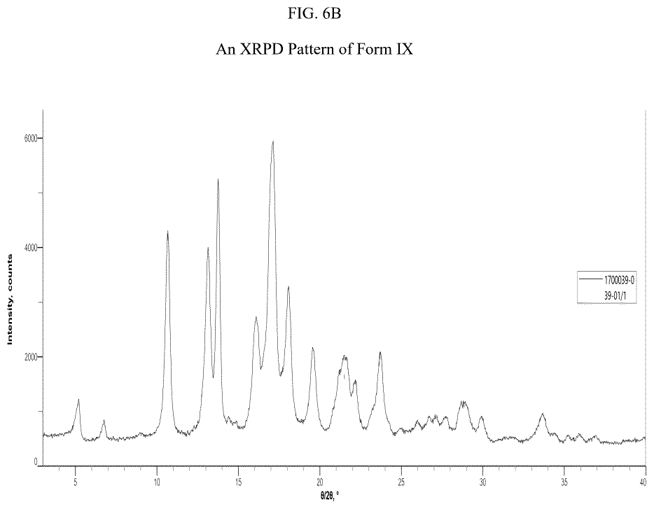

In other embodiments, crystalline Form IX has an XRPD pattern substantially as shown in FIG. 6A or FIG. 6B.

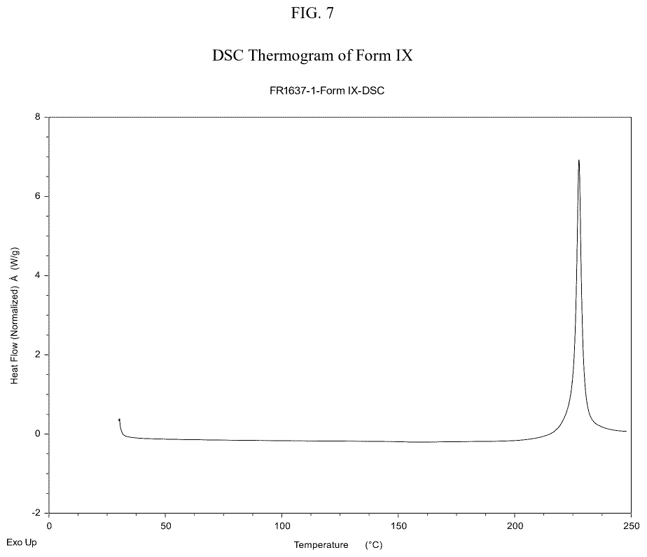

In one embodiment, crystalline Form IX is characterized by having a DSC thermogram substantially as shown in FIG. 7.

In another embodiment, crystalline Form IX has both: 1) a DSC characteristic described above; and 2) an X-ray powder diffraction pattern comprising at least one peak at °2θ selected from 5.5±0.3, 10.9±0.3, 14.0±0.3, 17.4±0.3, 21.7±0.3, and 24.0±0.3.

In another embodiment, crystalline Form IX has both: 1) a DSC characteristic described above; and 2) an XRPD pattern comprising peaks at °2θvalues of 5.5±0.3, 10.9±0.3, and 21.7±0.3.

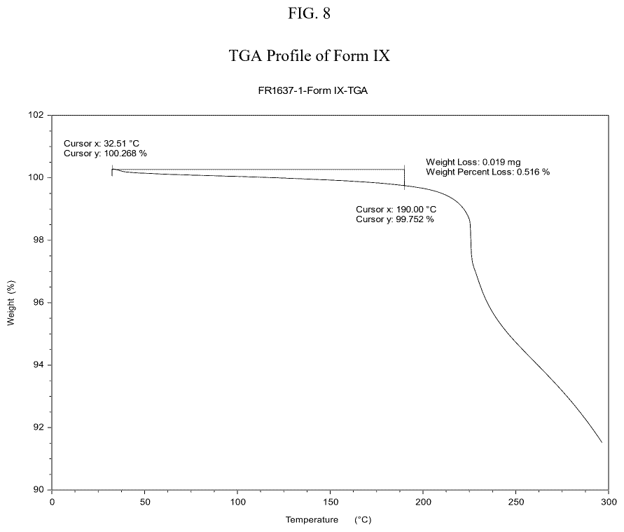

In one embodiment, crystalline Form IX is characterized by having about 0.5% weight loss until the onset of degradation at about 190° C. as estimated by TGA. In another embodiment, crystalline Form IX has a TGA profile substantially as shown in FIG. 8.

In another embodiment, crystalline Form IX has both: 1) one or more TGA characteristics described above; and 2) an XRPD pattern comprising at least one peak at °2θ selected from 5.5±0.3, 10.9±0.3, 14.0±0.3, 17.4±0.3, 21.7±0.3, and 24.0±0.3. In another embodiment, crystalline Form IX has both: 1) one or more TGA characteristics described above; and 2) an XRPD pattern comprising peaks at °2θvalues of 5.5±0.3, 10.9±0.3, and 21.7±0.3.

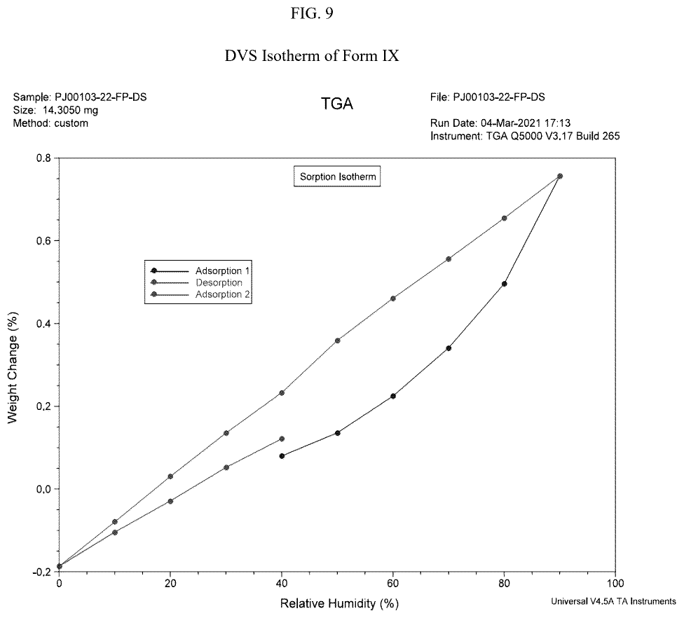

In one embodiment, crystalline Form IX has a DVS isotherm substantially as shown in FIG. 9.

In another embodiment, crystalline Form IX has both: 1) a DVS characteristic described above; and 2) an XRPD pattern comprising at least one peak at °2θ selected from 5.5±0.3, 10.9±0.3, 14.0±0.3, 17.4±0.3, 21.7±0.3, and 24.0±0.3.

In another embodiment, crystalline Form IX has both: 1) a DVS characteristic described above; and 2) an XRPD pattern comprising peaks at °2θvalues of 5.5±0.3, 10.9±0.3, and 21.7±0.3.

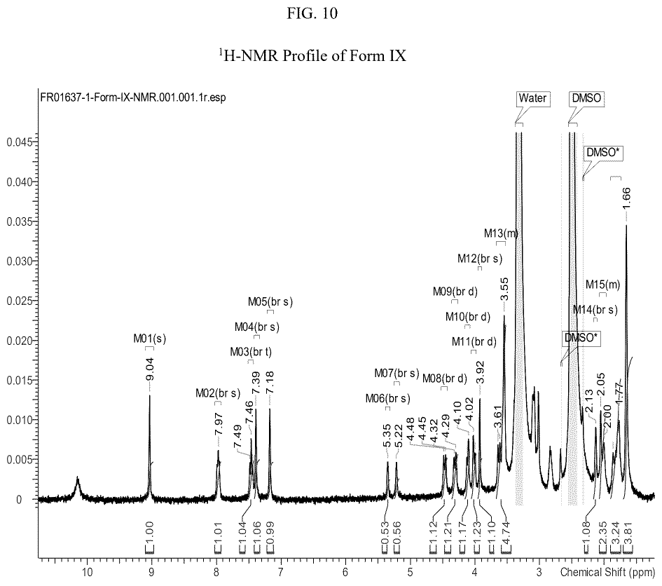

In one embodiment, crystalline Form IX has a ¹H-NMR profile substantially as shown in FIG. 10.

In one embodiment, crystalline Form IX is substantially free of residual organic solvents.

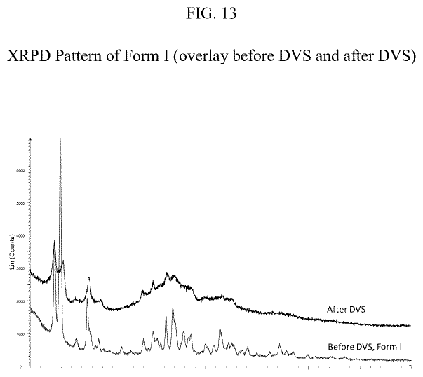

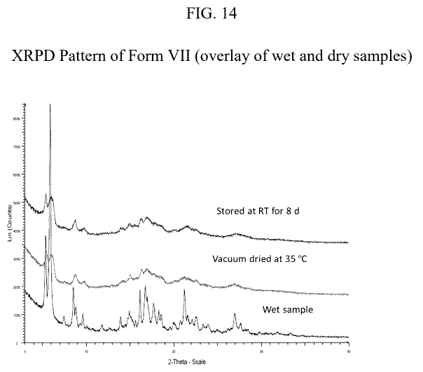

In other embodiments, the invention provides two more crystalline forms which are designated: Form II (hydrate) and Form VI (hydrate) and exist as families of solvates, hydrates, or hydrated solvates. There are also other crystalline forms of MRTX1133 that also exist as families of solvates, hydrates, or hydrated solvates, specifically, Form I, Form III, Form IV, Form V, Form and VII FIG. 13 illustrates an XRPD pattern of Form I, and FIG. 14 illustrates an XRPD pattern of Form VII.

However, Form VIII was selected as the development form.

In one embodiment, the crystalline forms of the present invention are at least 40%, 50%, 60%, 70%, 80%, 90% or 95% crystalline.

In another aspect of the invention, pharmaceutical compositions are provided for use in the methods comprising a therapeutically effective amount of at least one of the following: crystalline Form VIII and crystalline Form IX, and/or a pharmaceutically acceptable excipient. The invention also encompasses pharmaceutical compositions comprising any of other crystalline forms described in the application.

The invention also encompassed any mixtures of any of the described crystalline forms with the amorphous form of MRTX1133.

In some embodiments, the pharmaceutical compositions of the present invention comprise at least 95%, or at least 80%, or at least 70%, or at least 60%, or at least 50% of crystalline Form VIII of MRTX1133.

In some embodiments, the pharmaceutical compositions of the present invention comprise at least 95%, or at least 80%, or at least 70%, or at least 60%, or at least 50% of crystalline Form IX of MRTX1133.

In one aspect of the invention, provided herein are methods for inhibiting KRas G12D activity in a cell, comprising contacting the cell in which inhibition of KRas G12D activity is desired with a therapeutically effective amount of a crystalline form of the present invention, alone or in combination with one or more pharmaceutically acceptable excipients and/or diluents. In one embodiment, the crystalline form is crystalline Form VIII. In another embodiment, the crystalline form is crystalline Form IX. In other embodiments, the crystalline form is any of the other forms described in this application.

In one embodiment, the crystalline form is a mixture of crystalline Form VIII and crystalline Form IX. In another embodiment, the crystalline form is a mixture of any of the described crystalline forms with the amorphous form.

In one aspect of the invention, provided herein are methods of treating cancer in a subject in need thereof, comprising administering to the subject a therapeutically effective amount of a crystalline form of MRTX1133. In one embodiment, the cancer is a KRas G12D-associated cancer. In one embodiment, the KRas G12D-associated cancer is lung cancer.

In one embodiment, the crystalline form is crystalline Form VIII. In another embodiment, the crystalline form is crystalline Form IX. In other embodiments, the crystalline form is any of the other forms described in this application.

In one embodiment, the crystalline form is a mixture of crystalline Form VIII and crystalline Form IX. In another embodiment, the crystalline form is a mixture of any of the described crystalline forms with the amorphous form.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a crystalline form of MRTX1133, alone or in combination with one or more pharmaceutically acceptable excipients and/or diluent.

In one embodiment, the subject is an adult patient. In one embodiment, the subject is a pediatric patient.

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

In another aspect of the invention, provided herein are process for the preparation of crystalline forms of MRTX1133. In one embodiment, the process describes the preparation of crystalline Form VIII. In one embodiment, the process describes the preparation of crystalline Form IX. In other embodiments, the process describes the preparation of other crystalline forms of MRTX1133.

### Pharmaceutical Compositions

In another aspect, the invention provides pharmaceutical compositions comprising crystalline forms of MRTX1133 and a pharmaceutically acceptable carrier, excipient, or diluent that may be used in the methods disclosed herein. The crystalline forms of MRTX1133 may be formulated by any method well known in the art and may be prepared for administration by any route, including, without limitation, parenteral, oral, sublingual, transdermal, topical, intranasal, intratracheal, or intrarectal. In certain embodiments, the crystalline forms of MRTX1133 are administered intravenously in a hospital setting. In one embodiment, administration may be by the oral route.

In one aspect of the invention, pharmaceutical compositions are provided for use in the methods comprising a therapeutically effective amount of at least one of the following: crystalline Form VIII and crystalline Form IX, and/or a pharmaceutically acceptable excipient. The invention also encompasses pharmaceutical compositions comprising any of other crystalline forms described in the application.

The invention also encompassed any mixtures of any of the described crystalline forms with the amorphous form of MRTX1133.

In some embodiments, the pharmaceutical compositions of the present invention comprise at least 95%, or at least 80%, or at least 70%, or at least 60%, or at least 50% of crystalline Form VIII of MRTX1133.

In some embodiments, the pharmaceutical compositions of the present invention comprise at least 95%, or at least 80%, or at least 70%, or at least 60%, or at least 50% of crystalline Form IX of MRTX1133.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to about 300 mg/kg, from about 0.1 to about 100 mg/kg per day, from about 0.5 to about 50 mg/kg per day, or from about 1 to about 25 mg/kg per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art. The pharmaceutical compositions comprising the crystalline forms of MRTX1133 may be used in the methods of use described herein.

### METHODS OF USE

The compositions and methods provided herein may be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein are methods for treating cancer in a subject in need thereof, the method comprising (a) determining that cancer is associated with a SOS1 mutation (e.g., a SOS1-associated cancer) (e.g., as determined using a regulatory agency-approved, e.g., FDA-approved, assay or kit); and (b) administering to the patient a therapeutically effective amount of a crystalline form of MRTX1133, alone or in combination with one or more pharmaceutically acceptable excipients and/or diluent.

In one embodiment, the subject is an adult patient. In one embodiment, the subject is a pediatric patient.

In one embodiment, a crystalline form of MRTX1133 is administered as a capsule during the period of time. In embodiments of the invention, a tablet or capsule comprises about 10 mg to about 1500 mg, for instance about 10 mg, about 20 mg, about 30 mg, about 40 mg, about 50 mg, about 60 mg, about 70 mg, about 0 mg, about 90 mg, about 100 mg, about 125 mg, about 150 mg, about 175 mg, about 200 mg, about 250 mg, about 300 mg, about 350 mg, about 400 mg, about 500 mg, about 600 mg, about 700 mg, about 800 mg, about 900 mg, about 1000 mg, about 1100 mg, about 1200 mg, about 1300 mg, about 1400 mg and about 1500 mg.

In one embodiment, the method comprises oral administration of a crystalline form once or twice a day on a daily basis (during a period of time), e.g., in an amount of about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 250 mg, about 300 mg, about 350 mg, about 400 mg, about 500 mg, about 600 mg, about 700 mg, about 800 mg, about 900 mg, about 1000 mg, about 1100 mg, about 1200 mg, about 1300 mg, about 1400 mg and about 1500 mg. Oral administration of a crystalline form of MRTX1133 occurs, for example, once a day on a daily basis (during a period of time). In one embodiment, MRTX1133 is orally administered once daily. In one embodiment, the crystalline form of MRTX1133 is orally administered twice daily.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

The following Examples are intended to illustrate further certain embodiments of the invention and are not intended to limit the scope of the invention.

### Example 1

### Preparation of Crystalline Form VIII of MRTX1133

This Example illustrates the preparation of crystalline Form VIII of MRTX1133.
1. Charge MRTX1133 crude (380.0 g) and DCM (3.8 L) to a 50 L reactor (brown suspension).
2. Charge EtOH (3.8 L) to the reactor and stir for 1 h (Brown clear solution).
3. Charge 6% NaHCO₃ aq. (3.8 L) to the solution and stir for 0.5 h. Solid precipitation at the junction of organic phase and aqueous phase. Separation, the organic phase is filtered. Obtain organic phase 1 and aqueous phase 1.
4. The organic phase 1 is washed with 6% NaHCO₃ aq. (3.8 L) and stir for 0.5 h. Separation, obtain organic phase 2 and aqueous phase 2.
5 Combine the aqueous phase 1 and aqueous phase 2, then extract with DCM (3.8 L). Obtain organic phase 3 and aqueous phase 3 (discard).
6. Combine the organic phase 2 and organic phase 3, then wash with 10% NaCl aq. (3.8 L). Separation, obtain organic phase 4 and aqueous phase 4 (discard).
7. Charge 200-300 mesh silica (600 g) to chromatography column and rinse with DCM (3 L). Filter organic phase 4 through the silica gel column.
8. Rinse the silica gel column with 16 L (DCM:EtOH=4:1 v/v), obtain a light-yellow solution.
9. Concentrate to no fraction at 28° C. (a small amount of light-yellow solid precipitates).
10. Charge 7.6 L DCM to the concentrate and concentrate to no fraction (dark red solution).
11. Charge 7.6 L DCM to the concentrate and concentrate to 3-5 V (dark red solution).
12. Charge 7.6 L MTBE to the concentrate and concentrate to 3-5 V (dark red solution).
13. Charge 7.6 L MTBE to the concentrate and concentrate to 3-5 V (yellow solid precipitates).
14. Charge 19 L MTBE and concentrate (step 13) to a 50 L reactor.
15. Heat up to 50˜55° C. and stir for 12˜20 h.
16. Filter and the wet cake are washed 1.52 L*2 MTBE.
17. The cake is dried at 40˜50° C.
18. Obtained 236.8 g product as yellow solid.

### Example 2

### Preparation of Crystalline Form IX of MRTX1133

Charge 79.2 g of MRTX1133 free base in 60 mL DCM.
Adjust reactor temperature to 40° C. and stir for 0.5 h.
Add 60 mL MTBE into R1 over 1 h.
Adjust reactor temperature to 35° C.
Charge 0.2 g charcoal and stir for 0.5 h.
Filter warm solution (˜35° C.).
Wash with 10 mL DCM/MTBE (1:1 v/v).
Evaporated to dryness (light yellow solid).
Charge 30 mL MTBE and warm to 51° C.
Stir reactor for 99 h at 51° C. (same as 16 h).
Filter and wash with 20 mL MTBE (hot).
Dry for 20 h at 40° C. under vacuum.
Obtained 1.29 g of MRTX1133 Form IX with 97.8% purity.

### Example 3

### Preparation of Crystalline Form II of MRTX1133

Charge 500 mg of MRTX1133 sample in 2.5 mL EtOH/MCH (1:2 v/v) at 25° C. (sticky solid).
Stir sample for 48 h at 25° C.
Filter and dry for 54 h at 40° C. (no vacuum).
Obtained 340 mg of MRTX1133 Form II with 97.3% purity.

### Example 4

### Preparation of Crystalline Form VI of MRTX1133

Charge 300 mg of MRTX1133 sample in 2.5 mL MeOH
Adjust reactor temperature to 55° C. and stirred for 0.5 h at 55° C.
Adjust reactor temperature to 5° C. over 4 h.
Charge 5 mL isopropyl ether at 5° C. over 2 h.
Stir at 5° C. for 54 h.
Filter and dry for 54 h at 40° C. (no vacuum).

### Example 5

### Preparation of Crystalline Form I of MRTX1133

Form I was obtained by slurry experiments at RT in solvents of ethyl acetate (EA), isopropyl acetate (IPAC) and methyl ethyl ketone (MEK), or antisolvent crystallization in EA/Heptane, MEK/Heptane and acetone/EA, or slow evaporation in MEK. Form I presented rod-like shape. As 7.1% of EA was detected by ¹H-NMR for the sample obtained by slurry in EA, two stages of weight loss (6.6%) was observed by TGA from RT to 160° C. . . . The broad endothermic peak is spreading over 40-90° C. representing desolvation of EA. The exotherm could be assigned to melt and decomposition of the compound.

Form I could be obtained in various solvents. It is a family of isomorphic solvates. DVS diagram of Form I showed moisture content was increased with the increment of relative humidity from 0 to 90% RH. When equilibrated under 80% RH, the water content of Form I could reach as much as 10.3%. It is evident that the crystallinity of Form I dramatically decreased after DVS testing.

FIG. 13 illustrates an XRPD pattern of Form I (before and after DVS evaluation).

### Example 6

### Preparation of Crystalline Form VII of MRTX1133

Form VII was generated either by slow evaporation in variety of solvents, or slurry in MeOH/IPE at RT. XRPD patterns of Form VII and Form I were comparable. The crystallinity of Form VII was decreased after vacuum drying at 35° C. or placing at RT for 8 d.

FIG. 14 illustrates an XRPD pattern of Form I (wet and dried samples).

### Example 7

### Characterization of Crystalline Form VIII of MRTX1133

The starting material was characterized by XRPD, PLM, DSC, TGA, and ¹H-NMR. The results are summarized in Table 1.

TABLE 1
Characterization summary of MRTX1133 Form VIII

| Sample | Technique | Data | Results |
|---|---|---|---|
| MRTX1133 | XRPD | FIG. 1A | Highly crystalline, Form |
| Form VIII | | and Figure | VIII |
| | | 1B | |
| | PLM | | Fine-needle like particles |
| | | | and aggregates |
| | DSC | FIG. 2 | No melting point observed |
| | TGA | FIG. 3 | 0.7% weight loss observed |
| | | | before 180° C. |
| | DVS | FIG. 4 | 1.3% water uptake at |
| | | | 95% RH |
| | NMR | FIG. 5 | Residual solvent |

### Example 8

### Characterization of Crystalline Form IX of MRTX1133

The starting material was characterized by XRPD, PLM, DSC, TGA, and ¹H-NMR. The results are summarized in Table 2.

TABLE 2
Characterization summary of MRTX01133 Form IX

| Sample | Technique | Data | Results |
|---|---|---|---|
| MRTX1133 | XRPD | FIG. 6A | Highly crystalline, Form |
| Form IX | | and Figure | IX |
| | | 6B | |
| | PLM | | Fine-needle like particles |
| | | | and aggregates |
| | DSC | FIG. 7 | No melting point observed |
| | TGA | FIG. 8 | 0.5% weight loss observed |
| | | | before 190° C. |
| | DVS | FIG. 9 | |
| | NMR | FIG. 10 | No residual solvent |

### Example 9

### Characterization of Crystalline Forms II and VI of MRTX1133

TABLE 3
Characterization summary of crystalline Forms II and VI of MRTX1133

| | | Crystallinity | Wt. Loss in | Endo. in | Speculated |
|---|---|---|---|---|---|
| Crystal Form | | by XRPD | TGA (%, ° C.) | DSC | Form |
| MRTX1133 | Form II | High | 2.9 (100) | Dehydration | Hydrate |
| | | | | at ambient | |
| | | | | temperature | |
| | Form VI | High | 5.1 (120) | Dehydration | Hydrate |
| | | | | at ambient | |
| | | | | temperature | |

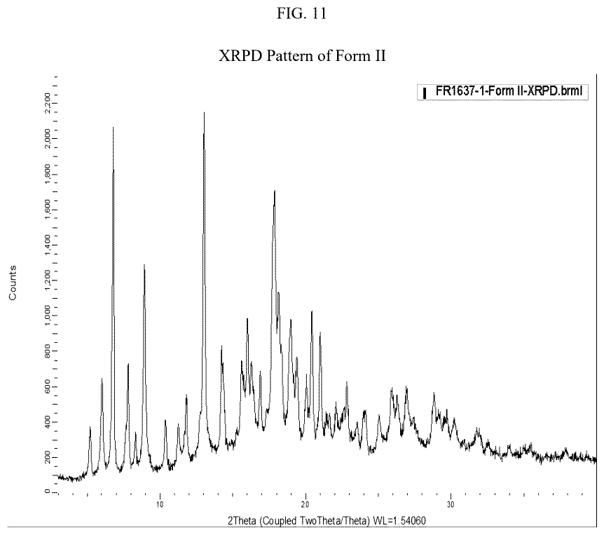

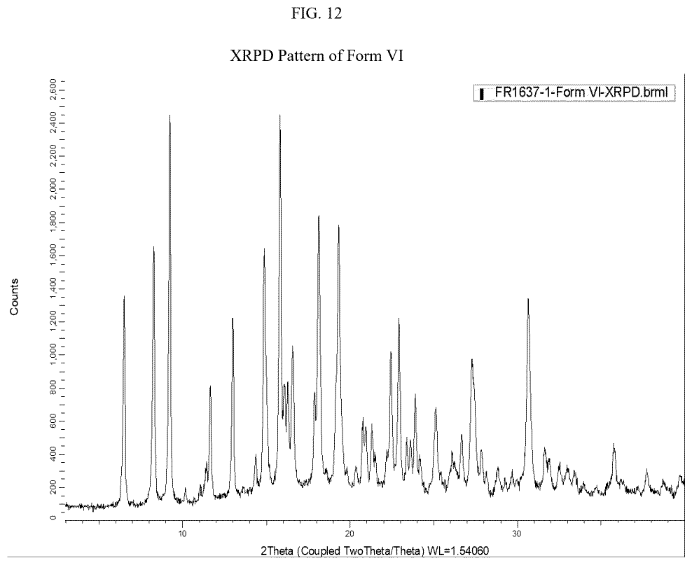

FIG. 11 illustrates an XRPD pattern of crystalline Form II prepared according to the methods described in the application. FIG. 12 illustrates an XRPD pattern of crystalline Form VI prepared according to the methods described in the application.

### Example 10

### Bulk Stability Studies of MRTX1133 Forms II, VI, VIII and IX

Stability studies of all four forms were conducted according to well-known methods. The results are summarized below in Tables 4-11.

TABLE 4
Solid State, 25° C./60% RH, open container, 1 week

| Parameter | Form II | Form VI | Form VIII | Form IX |
|---|---|---|---|---|
| Initial | 97.3% | 97.6% | 98.0% | 97.9% |
| Purity | | | | |
| by UPLC | | | | |
| Bulk | 97.4%; no | 97.7%; no | 98.0%; no | 98.0%; no |
| (UPLC) | change of | change of | change of | change of |
| | color | color | color | color |
| Bulk | Form II | No form | No form | No form |
| (XRPD) | with an | change | change | change |
| | additional | | | |
| | peak (may | | | |
| | belong to | | | |
| | Form IX) | | | |
| Morphology | Mainly block- | Columnar-like | Fine | Fine |
| | like particles | particles and | needle-like | needle-like |
| | with few | block-like | particles | particles |
| | columnar-like | particles | and some | |
| | particles | | aggregates | |

TABLE 5
Solid State, 25° C./92.5% RH, open container, 1 week

| Parameter | Form II | Form VI | Form VIII | Form IX |
|---|---|---|---|---|
| Initial Purity | 97.3% | 97.6% | 98.0% | 97.9% |
| by UPLC | | | | |
| Bulk (UPLC) | 97.8%; | 97.8%; no | 98.1%; no | 97.9%; no |
| | no | change of | change of | change of |
| | change of color | color | color | color |
| Bulk (XRPD) | Form II with an | No form | No form | No form |
| | additional peak | change | change | change |
| | (may belong to | | | |
| | Form IX) | | | |
| Morphology | Mainly block- | Columnar-like | Fine needle-like | Mainly fine |
| | like particles | particles and | particles and | needle-like |
| | with few | block-like | some aggregates | particles and |
| | columnar-like | particles | | few block-like |
| | particles | | | particles |

TABLE 6
Solid State, 40° C./75% RH, open container, 1 week

| Parameter | Form II | Form VI | Form VIII | Form IX |
|---|---|---|---|---|
| Initial Purity | 97.3% | 97.6% | 98.0% | 97.9% |
| by UPLC | | | | |
| Bulk (UPLC) | 96.7%; no | 97.6%; | 98.0%; no | 97.8%; no |
| | change of | no change | change of | change of |
| | color | of color | color | color |
| Bulk (XRPD) | Form II with an | No form change | No form change | No form change |
| | additional peak | | | |
| | (may belong to | | | |
| | Form IX) | | | |
| Morphology | Mainly block- | Columnar-like | Fine needle-like | Fine needle-like |
| | like particles | particles and | particles and | particles and |
| | with few | block-like | some aggregates | aggregates |
| | columnar-like | particles | | |
| | particles | | | |

TABLE 7
Solid State, 60° C., tight container, 1 week

| Parameter | Form II | Form VI | Form VIII | Form IX |
|---|---|---|---|---|
| Initial Purity | 97.3% | 97.6% | 98.0% | 97.9% |
| by UPLC | | | | |
| Bulk (UPLC) | 95.8%; no | 97.3%; no | 98.1%; | 97.9%; no |
| | change of | change of | no change | change of |
| | color | color | of color | color |
| Bulk (XRPD) | Form II with an | No form | No form | No form |
| | additional peak | change | change | change |
| | (may belong to | | | |
| | Form IX) | | | |
| Morphology | Mainly block- | Columnar-like | Fine | Mainly fine |
| | like particles | particles and | needle-like | needle-like |
| | with few | block-like | particles | particles and |
| | columnar-like | particles | | aggregates |
| | particles | | | |

TABLE 8
Solid State, 25° C./60% RH, open container, 2 weeks

| Parameter | Form VIII | Form IX |
|---|---|---|
| Initial Purity by | 98.0% | 97.9% |
| UPLC | | |
| Bulk (UPLC) | 98.0%; no change of color | 98.0%; no change of color |
| Bulk (XRPD) | No form change | No form change |
| Morphology | Fine needle-like | Mainly fine needle-like |
| | particles and | particles and few |
| | some aggregates | block-like particles |

TABLE 9
Solid State, 25° C./92.5% RH, open container, 2 weeks

| Parameter | Form VIII | Form IX |
|---|---|---|
| Initial Purity by | 98.0% | 97.9% |
| UPLC | | |
| Bulk (UPLC) | 98.0%; no change of color | 98.1%; no change of color |
| Bulk (XRPD) | No form change | No form change |
| Morphology | Fine needle-like particles | Fine particles and |
| | and some aggregates | aggregates |

TABLE 10
Solid State, 40° C./75% RH, open container, 2 weeks

| | | |
|---|---|---|
| Parameter | Form VIII | Form IX |
| Initial Purity by | 98.0% | 97.9% |
| UPLC | | |
| Bulk (UPLC) | 98.1%; no change of color | 98.0%; no change of color |
| Bulk (XRPD) | No form change | No form change |
| Morphology | Fine needle-like particles | Fine particles and |
| | and some aggregates | aggregates |

TABLE 11
Solid State, 60° C., tight container, 2 weeks

| | | |
|---|---|---|
| Parameter | Form VIII | Form IX |
| Initial Purity by | 98.0% | 97.9% |
| UPLC | | |
| Bulk (UPLC) | 98.1%; no change of color | 98.1%; no change of color |
| Bulk (XRPD) | No form change | No form change |
| Morphology | Fine needle-like particles and | Fine particles and |
| | some aggregates | aggregates |

### Example 11

### Stability Studies of Amorphous MRTX1133 and MRTX1133 Forms VIII and IX

Chemical stability and water content studies were conducted on amorphous MRTX1133 and MRTX1133 Forms VIII and IX according to well-known methods summarized below. The results are summarized below in Table 12.

TABLE 12
Accelerated stability study of MRTX1133 amorphous
form vs crystalline Forms VIII and IX

| | | | 25° C./60% RH | | | | 40° C./75% RH | | | |
|---|---|---|---|---|---|---|---|---|---|---|
| Material | Test | T(0) | 1M | 2M | 3M | 6M | 1M | 2M | 3M | 6M |
| Amorphous | Assay | 94.2^{b} | 89.5 | 87.7 | 86.1 | — | 82.8 | 70.1 | 61.3 | — |
| | (w/w %) | | | | | | | | | |
| | Total | 2.24 | 4.35 | 4.74 | 6.13 | — | 8.03 | 13.21 | 17.75 | — |
| | Impurities ≥ | | | | | | | | | |
| | 0.05% | | | | | | | | | |
| | (Area %) | | | | | | | | | |
| | Moisture, | 2.9 | 3.4 | 4.2 | 4.6 | — | 2.7 | 5.2 | 5.8 | — |
| | (% w/w) | | | | | | | | | |
| Form VIII | Assay | 96.9 | 96.8 | — | 97.3 | 96.4 | 96.6 | — | 97.4 | 96.1 |
| | (w/w %) | | | | | | | | | |
| | Total | 2.5 | 2.1 | — | 2.1 | 1.9 | 2.2 | — | 2.3 | 2.2 |
| | Impurities ≥ | | | | | | | | | |
| | 0.05% | | | | | | | | | |
| | (Area %) | | | | | | | | | |
| | Moisture, | 0.55 | 0.64 | — | 0.49 | 0.53 | 0.62 | — | 0.54 | 0.78 |
| | (% w/w) | | | | | | | | | |
| Form IX | Assay | 96.2 | 98.0 | 97.6 | 98.2 | 98.5 | 97.6 | 97.3 | 96.5 | 96.9 |
| | (w/w %) | | | | | | | | | |
| | Total | 1.2 | 1.5 | 1.4 | 1.7 | 1.7 | 1.8 | 1.8 | 2.0 | 2.3 |
| | Impurities ≥ | | | | | | | | | |
| | 0.05% | | | | | | | | | |
| | (Area %) | | | | | | | | | |
| | Moisture, | 0.94 | 0.56 | 0.65 | 0.70 | 0.66 | 0.56 | 0.63 | 0.66 | 0.70 |
| | (% w/w) | | | | | | | | | |

^{a} Assay and Total Impurities by HPLC unless specified otherwise
^{b}Assay by Q-NMR

The stability studies of the amorphous form were done as follows.

The required amount was placed in a clear Class A glass bottle, 24 mL, with closed-top cap, sealed in opaque resealable Zipper bag. Per each storage condition there was one bottle, and the sampling was performed from the same bottle. Each bottle was labeled with protocol number, compound ID, lot number and storage condition.

As Table 12 shows, for amorphous sample, there were substantial increases in impurity values and corresponding decreases in assay results for samples stored at all tested storage conditions. The most significant changes were noted at the 40° C./75% RH condition, where total impurities increased from 2.2% at T(0) to 13.2% at T(2M) to 17.8% at T(3M) and assay trended from 94% at T(0) to 70% at T(2M) to 61% at T(3M). Also, at the same 40° C./75% RH condition, moisture content increased notably from 2.9% at T(0) to 5.2% at T(2M), and then slightly increased to 5.8% at T(3M).

These results suggest that the amorphous form of MRTX1133 is not stable under the conditions tested.

In contrast, both crystalline Forms VIII and IX were shown to be much more stable under the same conditions.

The experimental conditions for stability testing of Forms VIII and IX were similar. However, the samples were placed in double LDPE bags filled with nitrogen protection, then placed into a heat-sealed foil bag, and into an HDPE container containing desiccant packs.

As Table 12 shows, for Forms VIII and IX, there were no increases in impurity values and no decreases in assay results for samples stored at all tested storage conditions.

At 40° C./75% RH condition, for Form VIII, total impurities went from 2.5% at T(0) to 2.2% at T(1M) to 2.3% at T(3M) to 2.2% at T(6M). Assay trended from 96.9% at T(0) to 96.6% at T(1M) to 97.4% at T(3M) to 96.1% at T(6M).

At 40° C./75% RH condition, for Form IX, total impurities went from 1.2% at T(0) to 1.8% at T(1M) and T(2M) to 2.0% at T(3M) to 2.3% at T(6M). Assay trended from 96.2% at T(0) to 97.6% at T (IM) to 97.3% at T(2M) to 96.5% at T(3M) to 96.9% at T(6M).

Also, at the same 40° C./75% RH condition, moisture content was relatively stable for both Form VIII and Form IX.

These results suggest that both Form VIII and Form IX are stable under the tested conditions.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A crystalline form of 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol.

2. The crystalline form according to claim 1, wherein the crystalline form is an anhydrate.

3. The crystalline form according to claim 1, wherein the crystalline form is Form VIII having an X-ray powder diffraction pattern comprising at least one peak at °2θ selected from 11.1±0.3, 13.4±0.3, 14.3±0.3, 19.1±0.3, 22.9±0.3, and 19.8±0.3.

4. The crystalline form according to claim 1, wherein the crystalline form is Form VIII having an X-ray powder diffraction pattern comprising peaks at °2θvalues of 11.1±0.3, 14.3±0.3, and 19.1±0.3.

5. The crystalline form according to claim 1, wherein the crystalline form is Form VIII having an X-ray powder diffraction pattern comprising two or more peaks at °2θ at 11.1±0.3, 13.4±0.3, 14.3±0.3, 19.1±0.3, 22.9±0.3, and 19.8±0.3.

6. The crystalline form according to claim 1, wherein the crystalline form is Form VIII having an X-ray powder diffraction pattern comprising three or more peaks at °2θ at 11.1±0.3, 13.4±0.3, 14.3±0.3, 19.1±0.3, 22.9±0.3, and 19.8±0.3.

7. The crystalline form according to claim 1, wherein the crystalline form is Form VIII having an XRPD pattern substantially as shown in FIG. 1A or FIG. 1B.

8. The crystalline form according to claim 1, wherein the crystalline form is Form VIII having a DSC thermogram substantially as shown in FIG. 2.

9. The crystalline form according to claim 1, wherein the crystalline form is Form VIII having a thermogravimetric analysis (“TGA”) profile substantially as shown in FIG. 3.

10. The crystalline form according to claim 1, wherein the crystalline form is Form VIII and which has about 0.7% weight loss until the onset of degradation at about 180° C. as estimated by TGA.

11. The crystalline form according to claim 1, wherein the crystalline form is Form VIII having dynamic vapor sorption (“DVS”) isotherm substantially as shown in FIG. 4.

12. The crystalline form according to claim 1, wherein the crystalline form is Form VIII which has an observed water uptake of about 1.3% at 25° C./95% Relative Humidity (RH), as measured by DVS.

13. The crystalline form according to claim 1, wherein the crystalline form is Form IX having an X-ray powder diffraction pattern comprising at least one peak at °2θ selected from 5.5±0.3, 10.9±0.3, 14.0±0.3, 17.4±0.3, 21.7±0.3, and 24.0±0.3.

14. The crystalline form according to claim 1, wherein the crystalline form is Form IX having an X-ray powder diffraction pattern comprising peaks at °2θvalues of 5.5±0.3, 10.9±0.3, and 21.7±0.3.

15. The crystalline form according to claim 1, wherein the crystalline form is Form IX having an X-ray powder diffraction pattern comprising two or more peaks at 5.5±0.3, 10.9±0.3, 14.0±0.3, 17.4±0.3, 21.7±0.3, and 24.0±0.3.

16. The crystalline form according to claim 1, wherein the crystalline form is Form IX having an X-ray powder diffraction pattern comprising three or more peaks at 5.5±0.3, 10.9±0.3, 14.0±0.3, 17.4±0.3, 21.7±0.3, and 24.0±0.3.

17. The crystalline form according to claim 1, wherein the crystalline form is Form IX having an XRPD pattern substantially as shown in FIG. 6A or FIG. 6B.

18-22. (canceled)

23. A pharmaceutical composition, comprising a therapeutically effective amount of a crystalline form of 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol according to claim 1.

24. The pharmaceutical composition according to claim 23, further comprising at least one pharmaceutically acceptable excipient and/or diluent.

25. A method for inhibiting KRas activity in a cell, comprising contacting the cell in which inhibition of KRas activity is desired with a therapeutically effective amount of a crystalline form according to claim 1, alone or in combination with one or more pharmaceutically acceptable excipient and/or diluent.

26. A method for treating cancer in a subject in need thereof comprising administering to the subject with a therapeutically effective amount of the crystalline form of 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol according to claim 1, alone or in combination with one or more pharmaceutically acceptable excipient and/or diluent.

27. The method according to claim 26, wherein the therapeutically effective amount of the crystalline form of 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol is between about 0.01 to 100 mg/kg per day.

28. The method according to claim 26, wherein the therapeutically effective amount of 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol is between about 0.1 to 50 mg/kg per day.

29. The method of claim 26, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

30. The method according to claim 26, wherein the cancer is a KRas G12C-associated cancer.

31. The method according to claim 26, wherein the cancer is non-small cell lung cancer.

32. The method according to claim 26, wherein the subject is an adult patient.

33. The method according to claim 26, wherein the subject is a pediatric patient.
